(19) United States
(12) Patent Application Publication
(10) Pub. No.: US 2008/0160603 A1
(43) Pub. Date: Jul. 3, 2008

(54) Flow stabilization in micro-and nanofluidic devices

(76) Inventors: Narayan Sundararajan, San Francisco, CA (US); Tae-Woong Koo, Cupertino, CA (US)

Correspondence Address: INTEL/BLAKELY, 1279 OAKMEAD PARKWAY, SUNNYVALE, CA, 94085-4040, US

(21) Appl. No.: 11/647,908

(22) Filed: Dec. 28, 2006

(51) Int. Cl. C12M 1/40; B01L 11/00; G01F 1/05

(52) U.S. Cl. 435/288.5; 137/115.01; 422/99

(57) ABSTRACT

Embodiments of the present invention provide microfluidic devices having deformable polymer membranes as components. The devices can be fabricated from a single polymeric block. Actuation of the membranes within the device allows the fluid contained within a microfluidic channel to be manipulated. Exemplary microfluidic devices, such as, peristaltic pumps, sample sorters, and flow stabilizers are described.

### CROSS REFERENCE TO RELATED APPLICATIONS

The present application is related to U.S. Patent Application Publication No. 2006/0073035, entitled “Deformable Polymer Membranes,” filed Dec. 30, 2004, now pending, the disclosure of which is incorporated herein by reference.

### BACKGROUND OF THE INVENTION

1. Field of the Invention

The embodiments of the present invention relate generally to microfluidic and nanofluidic devices and operations, deformable polymer membranes, and devices and methods for fluid flow and pressure stabilization.

2. Background Information

Micro-total-analytical systems (also known as lab-on-a-chip devices) are devices designed to miniaturize analytical or bioanalytical techniques and integrate them into a microfabricated format. Microfluidic and nanofluidic components for performing a variety of operations are integral parts of micro-total-analysis system applications. For example, cell sorters have become a vital component in micro total analysis systems aiming to investigate biological events at the single cell level. However it has not been easy to integrate different micro- and nano-fluidic components together into a single chip. This has been due to the different and sometimes difficult fabrication requirements for each of the micro- and or nano-fluidic components. For example, pumping in micro total analysis is generally achieved using external devices such as syringes or peristaltic pumps or using voltages across the channels generating electrokinetic or electroosmotic flow.

Essential processes such as bonding, aligning, clamping and interconnections for realizing a micro total analysis system generally cause significant device failure rates. Making components from the same basic unit and material facilitates the integration of operations and components. For example, polymers such as poly(dimethyl siloxane) (PDMS) can be used to fabricate various components in microfluidic devices. In addition, easy fabrication processes and simplicity of the device greatly help in integration of these components into a single device.

Flow control devices can be important components of lab-on-a-chip devices depending on the application and design of the chip. Often, a fluid entering a chip is mechanically pumped into the micro- or nano-channels of the chip. Depending on the pumping method or device chosen, significant fluctuations in both pressure and flow rate can occur in the micro- or nano-channels of the chip. Flow control devices are very useful for applications in which constant flow rate and or constant pressure are necessary, such as for example, controlled drug release, microreactors, microdialysis applications, chromatography, and proteomics applications.

### BRIEF DESCRIPTION OF THE FIGURES

FIGS. 1A and 1B provide schematics (a top view and a side view, respectively) of a deformable membrane operably connected to a microfluidic channel.

FIG. 2 shows a method for fabricating a microfluidic device using single layer soft lithography.

FIG. 3 shows simulations that have been performed to assess the flow stabilization characteristics of a deformable polymer membrane.

FIG. 4 shows simulations that have been performed to assess the flow stabilization characteristics of a series of deformable polymer membranes.

FIG. 5 provides two exemplary designs for peristaltic pumps incorporating deformable membranes.

FIGS. 6A, 6B, and 6C graph measurements of fluid flow rate versus the frequency of pressure applied to the operating channels for several peristaltic pump configurations.

FIGS. 7A and 7B show exemplary designs for microfluidic sorting devices.

### DETAILED DESCRIPTION OF THE INVENTION

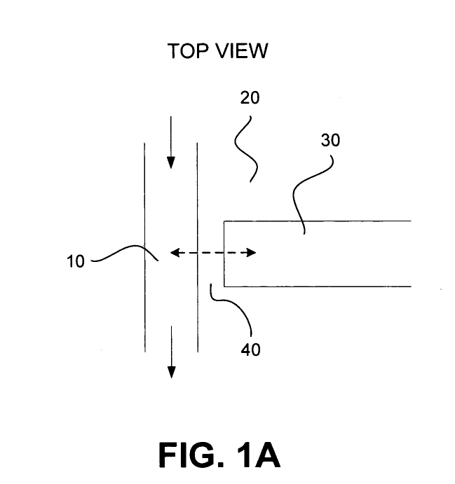

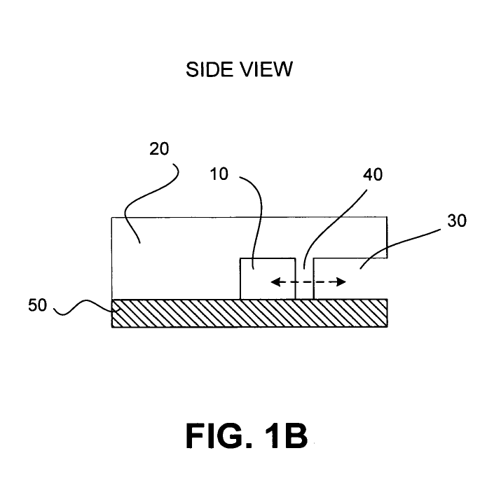

Embodiments of the present invention provide deformable polymer membranes as active components of a micro- and or nano-fluidic system. The deformable membranes perform functions associated with the manipulation of liquids in a micro- or nano-fluidic channel. Because the polymer membranes are disposed in the same polymer layer as the active microfluidic channel, manufacture of the microfluidic device is simplified. Although deformable membranes have been exemplified using PDMS, the present invention is not limited as other elasomeric polymers can be used to fabricate membranes. Using a deformable membrane unit such as that shown schematically in FIGS. 1A and 1B, consisting of an active microfluidic channel, an operating channel, and a membrane separating the channels, microfluidic components functioning, for example, as pumps, sorters, and mixers can be designed and fabricated.

In general, a microfluidic device comprises one or more channels having at least one dimension less than 1 mm and the device has the ability to support fluid flow within one or more channels. Nanofluidics refers to devices having channels that are about 100 to 1000 times smaller than microfluidic channels. The channels can be modified in numerous ways to accomplish various analytical tasks. Because the volume of fluids within microchannels is very small, usually several nanoliters or less, the amount of reagents and analytes used is small.

Referring to FIG. 1, a basic deformable membrane as part of a micro- or nano-fluidic device is illustrated. FIG. 1A provides a top-down view and FIG. 1B provides a side-view of the same section of a microfluidic device. A micro- or nano-fluidic channel 10 is formed in a solid polymer block 20. An operating channel 30 is operably connected to a membrane 40 that is formed by the intersection of the fluidic channel 10 and the operating channel 30 within the polymer block 20. As used herein, the term operating channels refers to channels that are operably connected to the deformable membrane to allow for deformation, actuation, and or pulsing of the membrane. The polymer block housing 20 is attached to a substrate 50. Actuation, deflection, or pulsing of the membrane 40 causes a change in the flow characteristics of the fluid contained within the fluidic channel 10. By placing the membrane in the same polymer layer as the micro- or nano-fluidic channel, device fabrication is facilitated.

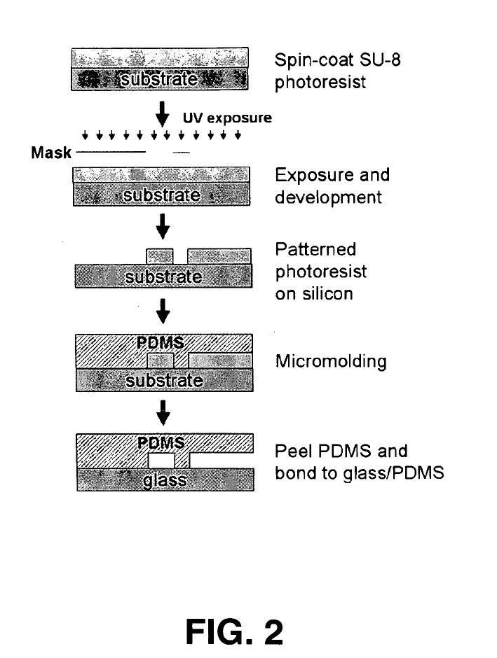

FIG. 2 provides a general outline for micro- or nano-fluidic chip fabrication using standard single layer soft lithography. In FIG. 2, a photoresist on silicon master is prepared using standard photolithography using a thick SU-8 photoresist spun at thickness of 100 μm. This is followed by micromolding with PDMS after which the PDMS mold is peeled off the master and bonded to a substrate, which for this example is a glass or PDMS surface. Other methods for forming microfludic structures are known and the invention is not limited to a particular method of forming the structures.

Embodiments of the present invention provide flow stabilization devices comprising deformable polymer membranes disposed along micro- or nano-fluidic channels. A flow stabilization device can comprise from 1 to thousands of deformable polymer membranes operably disposed in series along a micro- or nano-fluidic channel. The selection of the number of deformable polymer membranes in a flow stabilization device is a user-defined value, dependent in part on the surrounding devices in the micro- or nano-fluidic chip-based analysis application. The ease of integration of deformable polymer membranes according to embodiments of the present invention into micro- and nano-fluidic devices allows devices with 10 or more, 20 or more, 100 or more, or 500 or more deformable polymer membranes to be built. Additionally, methods for forming the deformable polymer membranes of the present invention provide highly miniaturized devices, even for devices having large numbers of deformable polymer membranes. Advantageously, a user can tune a device by selecting the number of deformable polymer membranes to be included to achieve a desired level of pressure and or flow rate fluctuation attenuation. Since the design of the deformable polymer membranes facilitates ease of fabrication, a miniaturized device having many closely spaced deformable polymer membranes may be fabricated. For example, the space between deformable polymer membranes may be 100 μm or less.

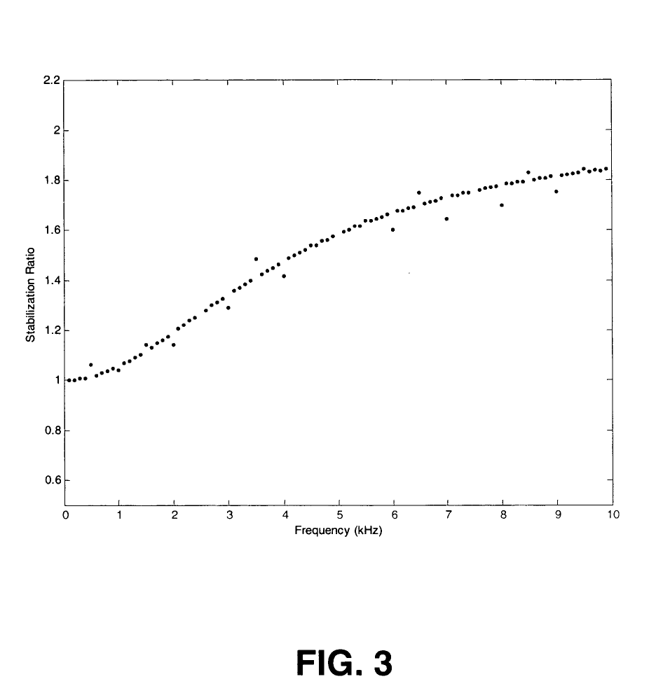

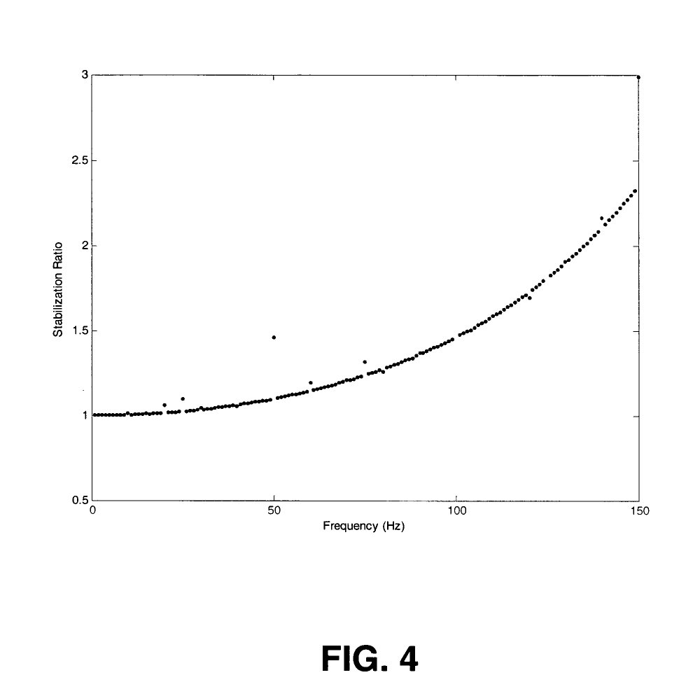

Simulations have been performed to assess the flow stabilizing characteristics of the deformable membrane units. FIG. 3 shows the flow-rate stabilization ratio (y-axis) of a microfluidic device employing one deformable polymer membrane versus the frequency of fluctuations in pressure and or flow rate (x-axis). In the simulation, the input and output flow rate had both a DC component and a sinusoidal AC component. The input flow rate (Qᵢ) can be represented by the equation Qᵢ=Q_{di}+Qₐᵢ sin(ωt+φ₁) where Q_{di} is the DC component of the input flow rate, Qₐᵢ is the amplitude of the AC component of the input flow rate, ω is the angular frequency, t is time, and φ₁ is the phase shift. The output flow rate (Qₒ) can be represented by the equation Qₒ=Q_{do}+Qₐₒ sin(ωt+φ₂) where Q_{do} is the DC component of the output flow rate, Qₐₒ is the amplitude of the AC component of the output flow rate, ω is angular frequency, t is time, and φ₂ is the phase shift. The flow-rate stabilization ratio is defined as Qₐₒ/Qₐᵢ. As can be observed in FIG. 3 the stabilization ration approaches the asymptote value of 2 at high frequencies (about 5 kHz). FIG. 4 shows the simulated flow-rate stabilization ratio (y-axis) of 625 deformable membrane units placed in series to form a microfluidic stabilizer chip. FIG. 4 demonstrates that the operating frequency greater than 180 Hz, a device comprising a plurality of deformable membranes provides more stabilization effect than a device comprising fewer deformable membranes.

Typically, methods for delivering fluids and reagents to a micro- or nano-fluidic device do not provide steady flow rates and or steady input pressures for the fluids and or reagents delivered. For example, flow delivery devices include, gravity devices, pumps, electrokinetic micropumps, peristaltic pumps, injectors, and syringes (both manually and mechanically driven). The sensitivity, accuracy, and or precision of many separation and analysis techniques can benefit from having steady pressures and flow rates for delivery of analytes. Typical separation and analysis techniques include, for example, high-performance liquid chromatography (HPLC), reversed phase HPLC, dialysis, electrophoresis, electrochromatography, and cell-sorters.

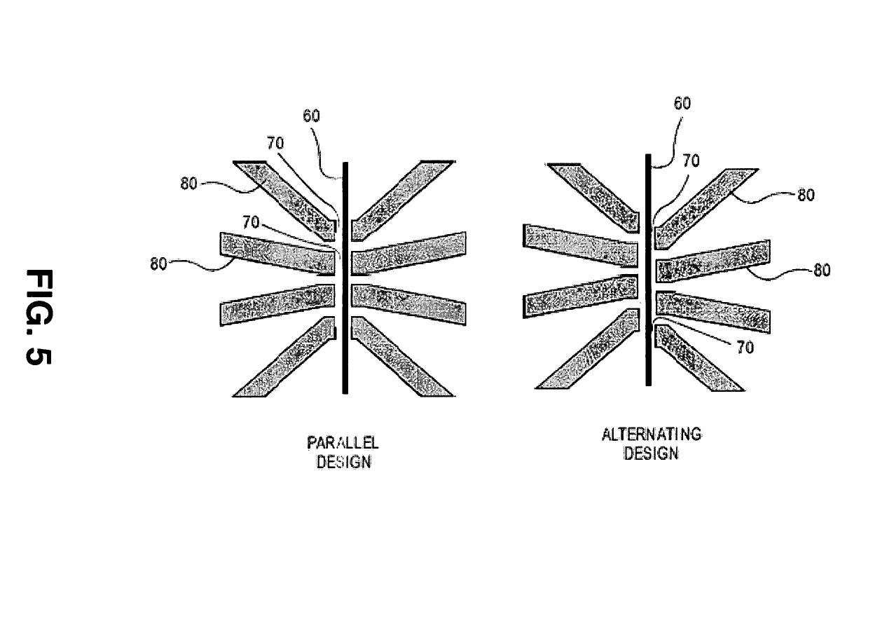

Peristaltic pumping of fluids within a microchannel can be effectuated using deformable membranes and operating channels that are disposed in the same polymer layer as the active microfluidic channel. The deformable membrane unit can be actuated, for example, by pressurizing the operating channels with a gas or liquid. Peristaltic pumps were realized by placing multiple deformable membrane units (a membrane unit is a pair of membranes disposed on opposite sides of a microfluidic channel) in series along a microfluidic channel. Referring now to FIG. 5, two different designs were built to compare pumping efficiency as a function of the placement of deformable membranes along a microfluidic flow channel 60. In one example, a symmetric parallel design had membranes 70 and operating channels 80 placed symmetrically on opposite sides of the microfluidic channel 60, as shown schematically in FIG. 5. In another example, an asymmetric alternating design had membranes 70 and operating channels 80 placed asymmetrically on each side of the fluid channel 60, as shown schematically in FIG. 5. In the example shown in FIG. 5, deformable membrane units 70 were staggered by 50 μm on opposite sides of the active microfluidic channel 60. Alternative dimensions are possible. The membranes on a side of the channel can be separated from each other, for example, by distances of about 200 μm to about 50 μm. Pumping was visualized using a diluted solution of 1 μm fluorescent poly(styrene) beads in water. Three different phase angles of actuation for the membranes, 60°, 90°, and 120° (corresponding to actuation patterns of (100, 110, 111, 011, 001, 000), (100, 110, 011, 001), and (101, 100, 110, 010, 011, 001), where 1 indicates the membrane is actuated (distended into the microfluidic channel) and 0 indicates the membrane is not actuated), were tested and it was found that for these designs, the 60° phase angle of actuation provided the fastest flow rate for both exemplary designs.

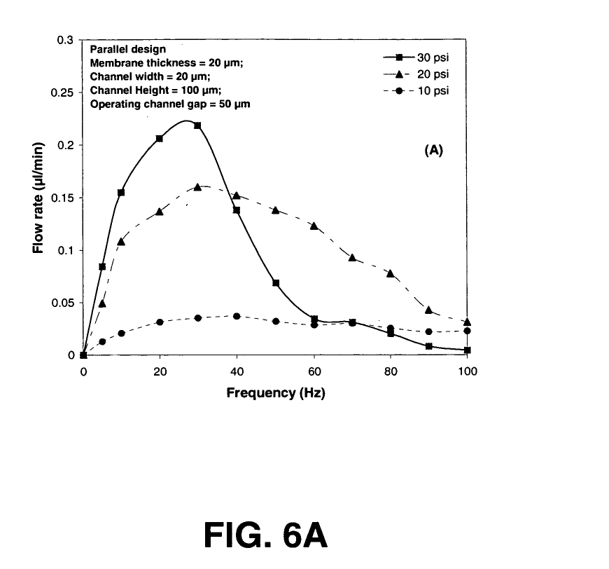

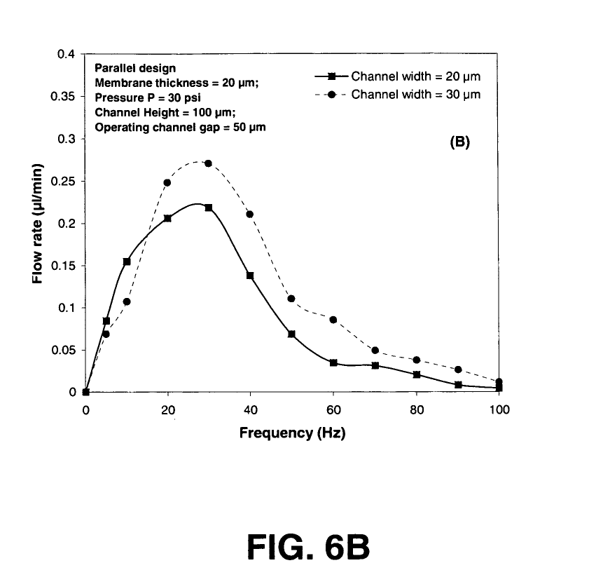

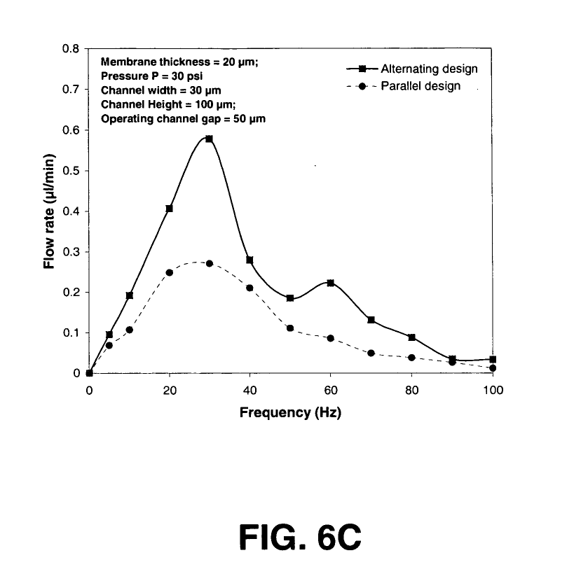

Several different parameters, including the external regulated pressure, frequency of actuation, microfluidic channel width, membrane thickness, channel height, and gap between air channels, were tested. Typical operating channel width was 100 μm. Flow rates were calculated by measuring the time taken for fluorescent beads to traverse through a 2.7 mm long serpentine channel. FIG. 6A shows the frequency dependence of flow rate for an exemplary parallel membrane device design (as diagrammed in FIG. 5) for different external applied pressures. The microfluidic device in this example had a membrane thickness of 20 μm, a microfluidic channel width of 20 m, a channel height of 100 μm, and an operating channel gap of 50 μm. As can be seen from the graph in FIG. 6A, the flow rate increases to a maximum at about 30 Hz and then drops down rapidly as frequency of actuation increases for all external pressures applied. It is believed that these results can be attributed to the spring force effect of the membrane in which, after a certain frequency, the membrane does not revert back to its original position thereby reducing the volume displacement of the fluid achieved. Also, as the external pressure applied is increased, the maximum flow rate obtained increases. It is believed that the increased external pressure applied to the membrane increases the deflection of the PDMS membrane thereby increasing the volume of the fluid displaced.

FIG. 6B shows the flow rate dependence at different frequencies of actuation for two exemplary parallel design devices (as diagrammed in FIG. 5) that had microfluidic channel widths of 20 μm and 30 μm. The devices had membrane thicknesses of 20 μm, channel heights of 100 μm, and operating channel gaps of 50 μm. The pressure applied to the operating channels was 50 psi. As seen from the graph in FIG. 6B, the trend of the flow rate dependence on the frequency of actuation is the same while the maximum flow rate obtained using the 30 μm width channel is higher than that of the 20 μm.

The results acquired from two exemplary designs for deformable membrane unit placement in a peristaltic pump (as shown in FIG. 5) are shown in FIG. 6C. In both cases, the dimensions of both the microfluidic and the operating channels were the same and the pressure applied was 30 psi. The membrane thickness was 20 μm, the microfluidic channel width was 30 μm, the microfluidic channel height was 100 μm, and the operating channel gap was 50 μm. As seen from the graph in FIG. 6C, the alternating design provided about twice the maximum flow rate of the parallel design. It was also found that the alternating design example prototype was only better at the higher pressure of 30 psi while the parallel design performed slightly better than the alternating design at pressures of 10 and 20 psi. It is believed that this observed enhancement can be attributed to the fact that in the alternating design, the deflection of the membrane is higher because it is not as constrained by the membrane on the other side of the microfluidic channel.

By controlling the various parameters of actuation and dimensions of the components of the basic deformable membrane unit, it is possible to control the flow velocities and rates. In general, channel aspect ratios of about 1:2 to about 1:10 (width to height) and widths of about 10 to about 100 μm have been used in embodiments of the present invention. Additionally, in general, average membrane thicknesses of about 5 to about 50 μm and distances between membranes located on a side of a channel of about 50 to about 200 μm can be used in embodiments of the present invention. The height and the width of the membranes are typically determined by the dimensions of the intersection of the microchannels that form the membranes which in turn are user-defined variables.

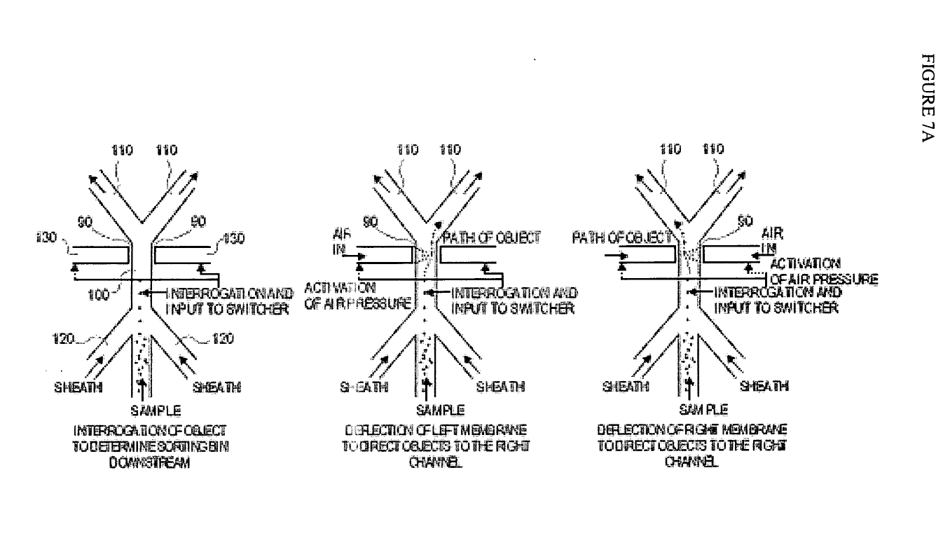

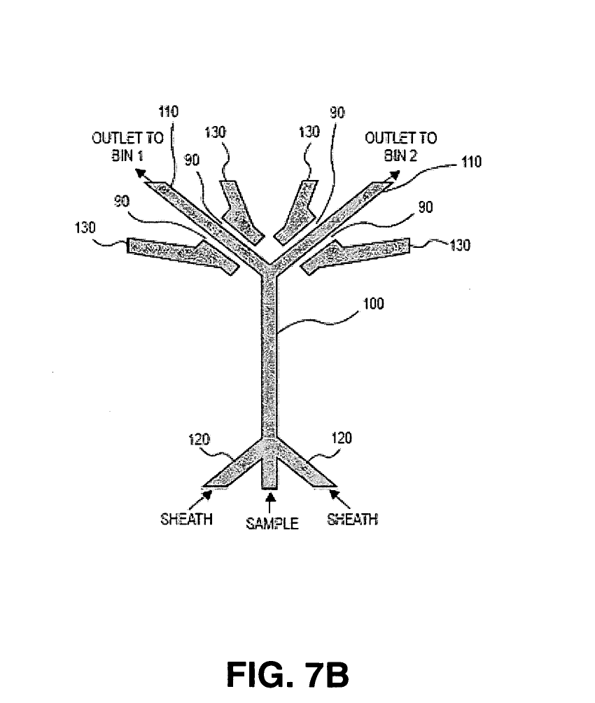

Referring now to FIG. 7, the placement of operating channels in several exemplary microfluidic sorting devices is diagrammed. In this example, deformable membranes 90 were placed along the main inlet microfluidic channel 100 or along each of the branch outlet microfluidic channels 110. The sample flow was hydrodynamically focused in the main microfluidic channel by using sheath flows from intersecting sheath microfluidic channels 120 on either side of the sample solution inlet channel 100. Two exemplary designs are shown: in FIG. 7A deformable membranes 90 are placed alongside the main inlet microfluidic channel 100, and in FIG. 7B deformable membranes 90 are placed alongside each of the branch microfluidic channels 110 (the branch channels are labeled “outlet to bin 1” and “outlet to bin 2” in FIG. 7B). In one embodiment, the membrane units 90 were activated by increasing air pressure in the operating channel 130 and causing the membrane 90 to deflect into the microfluidic channel 100 or 110. A diluted sample solution of 6 μm fluorescent poly(styrene) beads was hydrodynamically focused using branch sheath flows of DI water from either side of the main channel. To sort particles contained in a flow in the main microfluidic channel in an exemplary device having deformable membrane units placed alongside the main microfluidic channel, either the left or the right membrane is deflected into the channel to guide the microfluidic stream into the left or the right outlet channel, respectively. It was found that placement of the deformable membrane unit in the main microfluidic channel far from the Y-branch results in poorer sorting fidelity because of recovery of the laminar streams before reaching the Y-branch. Additionally, a sorting device may also optionally comprise a device for interrogating the sample stream and providing input to the switcher that activates the pressure in the operating channels. For example, the device for interrogating may be a UV-vis, fluorescence, or Raman detector that detects the presence of a cell, a virus, a bacterium, a label molecule, or a nanoparticle. When the detector detects a species of interest, it communicates to the switcher to direct the species into a selected outlet channel. For example, when the light intensity is above a certain threshold from a CCD camera used to detect a nanoparticle, the above-threshold signal can be converted using an algorithm to provide a voltage to the solenoid valves and cause a switcher to activate pressure in the operating channels.

In an exemplary design according to FIG. 7B, the deformable membrane units were placed in the branch channels that when actuated would increase the resistance to flow in the respective branch thereby diverting the direction of flow of the sample to the other branch. Six micrometer beads were sorted by using the deformable membrane units placed in a branch channel. The exemplary device had a microfluidic channel width of 100 μm, a channel height of 100 μm, a membrane thickness of 20 μm. Actuation of the deformable membranes in the right branch outlet directed the 6 μm bead to the left branch outlet. Actuation of the deformable membranes in the left branch outlet directed the 6 μm bead to the right branch outlet. This exemplary device design worked with approximately 100% fidelity for hydrodynamically focused beads, that is to say, approximately 100% of the beads went to the right branch when the deformable membrane on the left branch was actuated and vice versa.

The micro-fluidic channels represent micro-sized fluid passages that may have a cross-sectional dimensions, channel width, channel height, channel diameter, etc. that may be not greater than approximately one millimeter (mm, one-thousandth of a meter, also 1000 μm). In various embodiments the cross-sectional dimension may be not greater than approximately 500 micrometers (μm, one millionth of a meter), 200 μm, 100 μm, 50 μm, or 10 μm. The invention is not limited to any known minimum cross-sectional dimension for the channels. In various embodiments the cross-sectional dimension may be greater than approximately 0.001 μm (1 nm), greater than approximately 0.01 μm (10 nm), or greater than approximately 0.1 μm (100 nm). The optimal dimension of the channel may depend upon the characteristics of the fluids and or particles to be conveyed therein. An exemplary micro-fluidic channel which may be used for one or more of an inlet, outlet, or focusing channel, may comprise a rectangular channel having a channel width of approximately 100 μm and a channel height of approximately 50 μm. The rectangular shape and specific dimensions are not required. These miniaturized channels are often useful for handling small sized samples and allow many channels to be constructed in a small substrate, although this is not a requirement. There is no known minimum or maximum length for the channels. Commonly the channel lengths are at least several times their width and not more than several centimeters.

PDMS may offer certain advantages such as compatibility with biological materials and chemicals and transparency to facilitate alignment, although the use of PDMS is not required and other materials may optionally be employed for forming the housing containing the membranes and microchannels. Any machinable, etchable, reformable, moldable, stampable, embossable, or castable elastomeric material (a material that is capable of deforming when pressure is applied and returning to its original shape when pressure is removed) may potentially be used. In general, there are a wide variety of formulations for elastomeric polymers, and a choice of materials may be based upon considerations such as elasticity, gas and/or liquid permeability, cost of fabrication, and/or temperature stability. Suitable polymers include among others, polyurethanes, silicones, polybutadiene, polyisobutylene, polyisoprene, elastomeric formulations of polyvinylchloride, polycarbonate, polymethylmethacrylate, polytetrafluoroethylene (Teflon^{R}), and combinations of these materials. It may be appropriate to form focusing devices of polymers because these materials are inexpensive and may be injection molded, hot embossed, and cast.

In general, almost any non-absorbent material capable of presenting a smooth surface can be used to form the substrate. Possible substrates that could be used include glass; silicon; polymers, such as for example, PDMS, polystyrene, and polyethylene; silicon nitride; silicon dioxide; and metals, such as for example, gold, aluminum, and the like. The housing in which the channels and the membranes are formed may be reversibly or irreversibly attached to the substrate. For example, a PDMS housing can be reversibly attached to, for example, a PDMS or a glass surface through van der Waals forces. Additionally, adhesives such as silicone adhesives and epoxies can be used to bond the housing to the substrate. Choice of method of bonding is dependent in part on the materials chosen for the housing and the substrate, the desired user-chosen operating pressure ranges, and functional compatibility with operating fluids chosen for a particular application and can be effectuated according to well-known methods in the art. Additionally, PDMS, for example, can be oxidatively sealed to, for example, PDMS, silicon, polystyrene, polyethylene, silicon nitride, or glass by exposing the surfaces to be bonded to an air plasma and bringing the surfaces into contact within about a minute after oxidation.

The invention is generally not limited to any known process flow. Suitable process flows may comprise an aqueous, organic, or biological solution. The process flow may contain a species of interest. The species of interest may comprise a biological material, such as a cell, organelle, liposome, biological molecule or macromolecule, enzyme, protein, protein derivative, protein fragment, polypeptide, nucleic acid, DNA, RNA, nucleic acid derivative, biological molecule tagged with a particle, fluorescently labeled biological molecule, charged species, or charged protein. Additionally, a process flow may contain reagents for chemical reactions and the products of chemical reactions.

Further, the deformable membranes can be actuated (deflected) pneumatically, hydraulically, piezoelectrically, thermopneumatically, and magnetically. Pneumatic and hydraulic actuation can be accomplished by pumping a gas or liquid, respectively, into an operating channel. Typically, the gas or liquid can be supplied and vented through a valve that is controlled by a valve drive and a computer generating a programmed actuation pattern that is converted into a control signal. Piezoelectric disks are commercially available from, for example, Piezo Systems, Inc (Cambridge, Mass.).

### EXAMPLES

Precursors for poly(dimethyl siloxane), Sylgard A and B were obtained from Dow Corning Inc. 1 and 6 μm YG fluorescent poly(styrene) beads used to visualize flow were obtained from Polysciences Inc. SU-2035 Photoresist was obtained from Microchem Corp.

An actuation system consisting of hardware and software components was constructed for pneumatically controlling the operating channels. The actuation system consisted of a control computer generating a programmed actuating pattern that was converted into a control signal through a digital output board (NI MIO-16XE-10, National Instruments). The control signal operated the valve drive (NI SCCDO01, National Instruments) that converted the control signals into the appropriate power leveled operating power patterns for switching the solenoid valves (LHDA1223111H, Lee company). Regulated external gas pressures (10-30 psi) were provided to the normally closed port of the manifold on which the solenoid valves were mounted allowing the operating channels to be pressurized or vented.

The valve drives were enclosed in the signal conditioning box (NI SCC2345, National Instruments) having two RJ45 connectors, two sets of banana connectors and four LEDs. Two sets of banana connectors provided the external power which then was converted into the pulsing power by valve drives. There were eight valve drives and each set of banana connector was connected to four valve drives so that enough external power was supplied. Two 12 V power supplies were connected to the banana connectors. The role of valve drive was to turn on and off the external power for solenoid valves so that it generated the patterned pulsing power with particular frequencies.

The application for the actuation system was written in C language. In order to increase the response time to maximum, Graphic User Interface (GUI) was not implemented. Actuation patterns for performing synchronized actuation of the different deformable membrane units were implemented in the software depending on the microfluidic operations.

Designs of the micro fluidic channels to be fabricated were drawn to scale using L-Edit (Tanner Research) and chrome masks were printed using a Micronics laser writer at Stanford nanofabrication facility.

SU-8 2035 photoresist was spun onto 4” silicon wafers at 2000 rpm for 30 sec. The wafers were then baked at 65° C. for 6 min. and at 95° C. for 20 min. The wafers are then exposed using UV light (365 nm) at a dose of about 400 mJ/cm². The exposed wafers were then baked at 65° C. for 1 min and at 95° C. for 5 min. After post-exposure bake, the wafers were immersed in SU-8 developer for about 10 min. to develop the unexposed regions. The SU-8 photoresist on the wafer was then silanized for 1 hr by placing the wafers in close proximity with a few drops of trimethylchlorosilane in a vacuum desiccator. The silanized photoresist on the wafer was used as the master for subsequent micromolding experiments.

Ten parts by weight of Sylgard A were added to 1 part by weight of Sylgard B, mixed thoroughly and degassed to remove any air bubbles to form the PDMS precursor. PDMS precursor was poured onto the silanized master and then cured at 65° C. for 1 hr. The cured PDMS was peeled off the master and holes were punched for reservoirs. In order to irreversibly seal the PDMS to a glass cover, the PDMS and the glass cover were placed in a plasma cleaner and treated with plasma (100 W) generated from ambient air for 1 min. and brought into conformal contact within 30 sec.

What is claimed is:

1. A device comprising:
a housing formed from a unitary section of polymer;
at least one microfluidic channel formed in the unitary section of polymer;
at least 10 deformable polymer membranes operably coupled to the microfluidic channel, wherein the deformable polymer membranes are formed from the unitary section of polymer, wherein the deformable polymer membranes have two surfaces, one surface that faces into the microfluidic channel and one surface that faces into a second channel, and wherein the deformable polymer membranes are disposed in series along the microfluidic channel; and
a solid substrate having a surface to which the housing is attached.

2. The device of claim 1 wherein the device comprises at least 100 deformable polymer membranes.

3. The device of claim 1 wherein the device comprises at least 500 deformable polymer membranes.

4. The device of claim 1 wherein a distance separating a first deformable polymer membrane from a second deformable polymer membrane is 100 μm or less.

5. The device of claim 1 wherein the microfluidic channels are nanofluidic channels.

6. The device of claim 1 wherein the polymer is selected from the group consisting of polyurethanes, silicones, polybutadiene, polyisobutylene, polyisoprene, elastomeric formulations of polyvinylchloride, polycarbonate, polymethylmethacrylate, polytetrafluoroethylene, and poly(dimethyl siloxane).

7. The device of claim 1 wherein the device additionally comprises a mechanical fluid delivery device operably connected to the microfluidic channel.

8. The device of claim 1 wherein the device additionally comprises a region through which fluid can flow comprising chromatographic separation media.

9. The device of claim 1 wherein the substrate surface is a material selected from the group consisting of glass, plastic, poly(dimethyl siloxane), metal, silicon nitride, silicon dioxide, and silicon.

10. The device of claim 1 wherein the device additionally comprises a cell sorter operably coupled to the microfluidic channel.

11. A method for stabilizing flow in a microfluidic channel comprising,
providing a housing formed from a unitary section of polymer having a microfluidic channel formed within the housing, the microchannel having at least 5 deformable polymer membranes operably coupled to the microfluidic channel, wherein the deformable polymer membranes are formed from the unitary section of polymer, wherein the deformable polymer membranes have two surfaces, one surface that faces into the microfluidic channel and one surface that faces into a second microchannel, wherein the deformable polymer membranes are disposed in series along the microfluidic channel, and wherein the housing is attached to a solid substrate;
flowing a liquid through the microfluidic channel wherein the flow rate of the liquid entering the channel varies over time; and
flowing the liquid past the at least 5 deformable polymer membranes in a manner that allows the variation in liquid flow rate to be attenuated.

12. The method of claim 11 wherein the device comprises at least 50 deformable polymer membranes.

13. The method of claim 11 wherein the device comprises at least 100 deformable polymer membranes.

14. The method of claim 11 wherein the device comprises at least 500 deformable polymer membranes.

15. The method of claim 11 wherein a distance separating a first deformable polymer membrane from a second deformable polymer membrane is 100 μm or less.

16. The method of claim 11 wherein the microfluidic channel is a nanofluidic channel.

17. The method of claim 11 wherein flowing a liquid comprises mechanically pumping the liquid.

18. The method of claim 11 additionally including flowing the liquid through a chromatographic separation media.

19. The method of claim 11 additionally including flowing the liquid through a cell sorter.
